(19) United States
(12) Patent Application Publication
(10) Pub. No.: US 2021/0196166 A1
(43) Pub. Date: Jul. 1, 2021

(54) METHOD AND TOOL SET FOR COLLECTING BODY FLUID OF A PERSON TO BE EXAMINED

(71) Applicant: TAI-SAW Technology Co., Ltd., Taoyuan City (TW)

(72) Inventors: Wei-Yi Hsu, Taoyuan City (TW); Pei-Tzu Hung, Taoyuan City (TW); Yi-Qi Huang, Taoyuan City (TW); Szu-Heng Liu, Taoyuan City (TW); Yu-Tung Huang, Taoyuan City (TW)

(73) Assignee: TAI-SAW Technology Co., Ltd., Taoyuan City (TW)

(21) Appl. No.: 16/990,595

(22) Filed: Aug. 11, 2020

(30) Foreign Application Priority Data
Dec. 31, 2019 (TW) 108148734

(51) Int. Cl. A61B 5/15; A61B 10/00

(52) U.S. Cl. CPC A61B 5/150358; A61B 5/150755; A61B 10/0051; A61B 5/150099; A61B 2010/0067; A61B 2010/008; A61B 10/007

(57) ABSTRACT

A method for collecting a body fluid sample of a person to be examined is provided. The method comprises steps of: providing a body fluid sample collecting device and a body fluid sample inspection device for collecting the body fluid sample according to a test sampling requirement, wherein the body fluid sample collecting device includes a body fluid sample collecting region having an open hydrophilic layer with a specific layer area, and configured for collecting a predetermined amount of the body fluid sample; from a body surface of the person to be examined, causing the body fluid sample to be collected onto the body fluid sample collecting region until the body fluid sample covers the hydrophilic layer completely; and receiving the body fluid sample from the body fluid collecting region to be inspected in the body fluid sample inspection device.

### CROSS-REFERENCE TO RELATED APPLICATION AND CLAIM OF PRIORITY

The application claims the benefit of Taiwan Patent Application No. 108148734, filed on Dec. 31, 2019, at the Taiwan Intellectual Property Office, the disclosures of which are incorporated herein in their entirety by reference.

### FIELD OF THE INVENTION

The present invention is related to methods and a tool set for collecting a body fluid sample, and more particularly to methods and a tool set for collecting a body fluid sample of a person to be examined.

### BACKGROUND OF THE INVENTION

Dropper-typed body fluid sample collection tools are often used to collect blood from the body surface. In order to make fresh blood samples available for testing as soon as possible, a conventional technical solution is to directly arrange biochemical reagents into the sample channel, chamber and microfluidic channel of the dropper. As shown in FIG. 1, the body fluid sample collection tool 10 includes a channel 12 and a biochemical reagent 14 disposed in a middle portion of the channel 12. When a user uses the body fluid sample collection tool 10 to approach a patient's body surface 2 to collect the body fluid sample 1 (for example, blood) on the body surface 2, the body fluid sample 1 sucked into the channel 12 can be advanced toward the right side in the figure. Eventually, the body fluid sample 1 will reach the position where the biochemical reagent 14 is arranged in the middle of the channel 12, and react with the biochemical reagent 14. Therefore, during the process of collecting body fluid samples, the required biochemical analysis starts simultaneously.

However, the above-mentioned conventional techniques have a potential risk of infection. To put it simply, it is often inevitable that a wound will be caused on the skin surface of the patient so that the blood sample can be collected from the wound when the blood sample is to be collected from the patient. As shown in the figure, the body fluid sample 1 can flow directly to the position of the biochemical reagent 14 through the channel 12, and on the contrary, once the biochemical reagent 14 contacts the body fluid sample 1, it may be transferred to the patient's body surface 2, or even pass through the wound (not shown) into the patient's body. Certain diseases such as mad cow disease can be transmitted through the above-mentioned ways. In addition, for many biochemical tests, the amount of body fluid samples required for each examination is related to the concentration or dose of biochemical reagents in the device. The above-mentioned conventional techniques cannot provide quantitative body fluid samples for analysis.

Therefore, how to avoid the above disadvantages is a technical problem that needs to be solved.

### SUMMARY OF THE INVENTION

In accordance with one aspect of the present invention, a method for collecting a body fluid sample of a person to be examined is provided. The method comprises steps of: providing a body fluid sample collecting device and a body fluid sample inspection device for collecting the body fluid sample according to a test sampling requirement, wherein the body fluid sample collecting device includes a body fluid sample collecting region having an open hydrophilic layer with a specific layer area, and configured for collecting a predetermined amount of the body fluid sample; from a body surface of the person to be examined, causing the body fluid sample to be collected onto the body fluid sample collecting region until the body fluid sample covers the hydrophilic layer completely; and receiving the body fluid sample from the body fluid collecting region to be inspected in the body fluid sample inspection device.

In accordance with another aspect of the present invention, a kit for collecting a body fluid sample of a person to be examined is disclosed. The kit comprises a body fluid sample collecting device and a body fluid sample inspection device. The body fluid sample collecting device includes a body fluid sample collecting region, wherein the body fluid sample collecting region has an open hydrophilic layer with a specific layer area, and is configured for collecting a predetermined amount of the body fluid sample. The body fluid sample inspection device includes a sampling inlet, a microfluidic portion connected to the sampling inlet and a reagent disposed on the microfluidic portion, and is configured to receive therein for inspection the body fluid sample from the body fluid sample collecting region.

In accordance with yet another aspect of the present invention, a method for collecting a body fluid sample of a person to be examined is disclosed. The method comprises steps of: selecting a body fluid sample collecting device and a body fluid sample inspection device for collecting the body fluid sample according to a test sampling requirement, wherein the body fluid sample collecting device includes a body fluid sample collecting region having an open hydrophilic layer with a specific layer area, and configured for collecting a predetermined amount of the body fluid sample; from a body surface of the person to be examined, causing the body fluid sample to be collected onto the body fluid sample collecting region until the body fluid sample covers the hydrophilic layer completely; and receiving the body fluid sample from the body fluid collecting region to be inspected in the body fluid sample inspection device.

The kit and method for collecting bodily fluid samples of a person to be tested provided by the present invention use the simplest method to achieve the effect of quantitative collection, and completely avoid the problem of the person being infected by chemical reagents, which is in line with the needs of the biotechnology industry. Thus, the present invention has utility for industry.

The objectives and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 is a schematic diagram showing a body fluid sample collection tool and its operation method according to the prior art;

FIG. 2A is a schematic diagram showing the first step of the method of using the kit for collecting a body fluid sample of a person to be examined according to one embodiment of the present invention;

FIG. 2B is a schematic top view diagram showing the second step of the method of using the kit for collecting a body fluid sample of a person to be examined according to one embodiment of the present invention

FIG. 2C is schematic side view diagram showing the embodiment according to the second step of the method as shown in FIG. 2B;

FIG. 3 is a schematic diagram showing various aspects of using the body fluid collecting device with opened hydrophilic layer for collecting body fluid sample from the person to be examined;

FIG. 4 is a schematic diagram showing the combination between the body fluid collecting device, body fluid sample inspection device and the biosensor for collecting body fluid sample from the patient to be examined; and

FIG. 5 is a schematic flow diagram showing the method of using the kit for collecting a body fluid sample of a person to be examined according to one embodiment of the present invention.

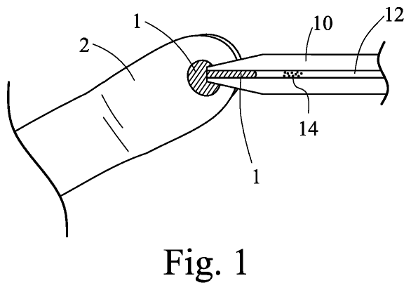

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; they are not intended to be exhaustive or to be limited to the precise form disclosed.

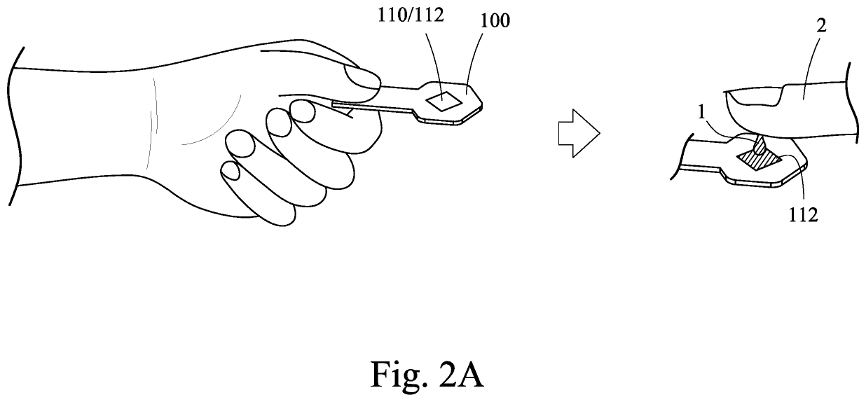

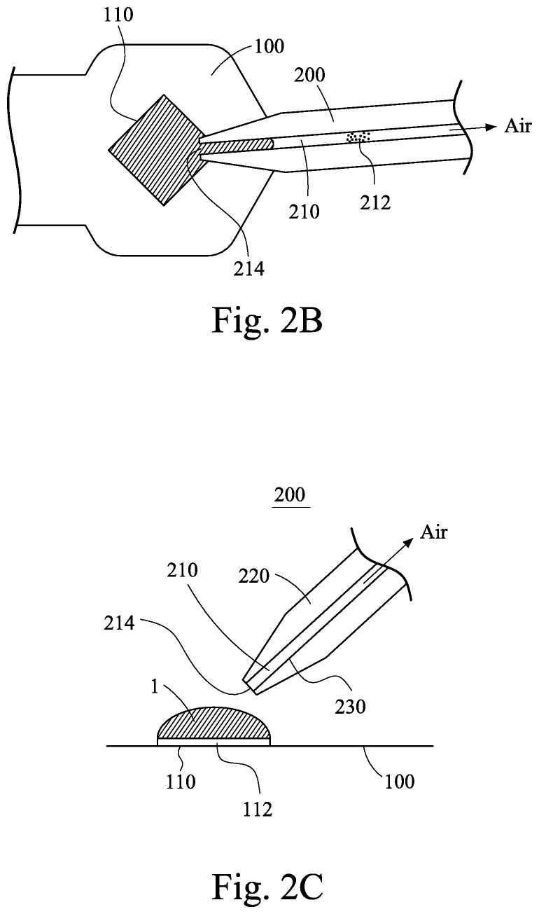

Please refer to FIG. 2A, which illustrates an embodiment of a body fluid sample collecting device 100 and a method of using the body fluid sample collecting device 100 in a set for collecting a body fluid sample of a person to be examined according to the present invention. The body fluid sample collecting device 100 can be made into a small apparatus that is easy to carry, and at least one body fluid sample collecting region 110 having an open hydrophilic layer 112 is disposed thereon.

FIG. 2A shows a diamond-shaped body fluid sample collecting region 110 as an example. Because the hydrophilic layer 112 has a hydrophilic property, the body fluid sample 1 is easily affected by the hydrophilic property of the hydrophilic layer 112 to flow in and fill the body fluid sample collecting region 110 when any side or corner of the body fluid sample collecting region 110 is close to the body surface carrying the body fluid sample 1 of the person to be examined. In addition, the hydrophilic layer 112 has a specific area, so that the amount of body fluid sample 1 on the body fluid sample collecting region 110 becomes a constant amount when the body fluid with specific viscosity properties fills the hydrophilic layer 112.

Please refer to FIGS. 2B-2C. After using the bodily fluid sample collecting region 110 on the bodily fluid sample collecting device 100 to collect a sufficient amount of the bodily fluid sample 1, one may select a specific chemical reagent 212 in the microfluidic channel 210 according to the requirements of the examination. The body fluid sample inspection device 200 is configured to collect and store the body fluid sample 1. The types of the chemical reagent 212 include antibodies, enzymes, proteins, any infectious substances, toxic chemicals or heavy metals. During the storage process, the body fluid sample 1 can react with the chemical reagent 212. After a certain period of time, the reaction is completed, and an appropriate biosensing device (not shown) can be used to identify the test result. After the reaction of some chemical reagents 212 and body fluid sample 1 is completed, the test results can even be visually identified without the aid of a biosensing device.

In the embodiment shown in FIG. 2C, the body fluid sample inspecting device 200 includes an upper-layer plate 220, a lower-layer plate 230 and a microfluidic channel 210 formed therebetween. The body fluid sample 1 can enter the microfluidic channel 210 through the capillary phenomenon, or according to an embodiment, an extraction device (not shown) is arranged at the other end of the microfluidic channel 210 to accelerate the flow of the body fluid sample 1 in the microfluidic channel 210.

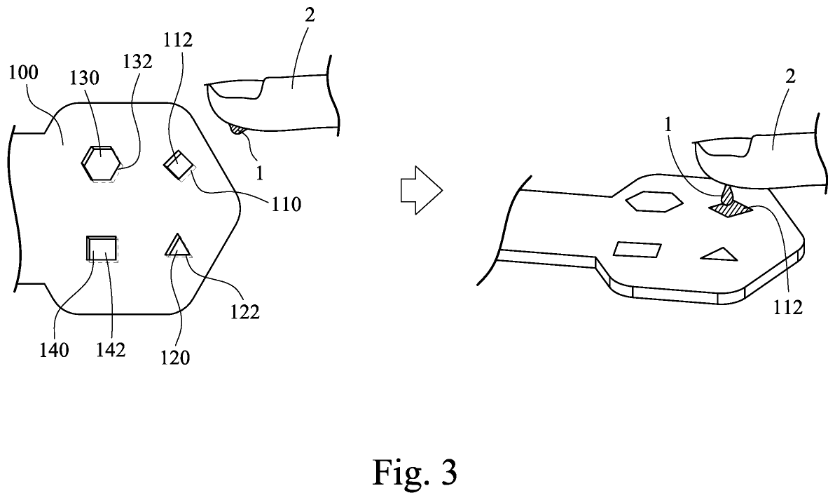

Please refer to FIG. 3, according to an embodiment of the present invention, the body fluid sample collecting device 100 may be configured with other different body fluid sample collecting regions 110/120/130/140, each of which has a unique shape and area. Since the open hydrophilic layer 112/122/132/142 is arranged on these body fluid sample collecting regions 110/120/130/140 in an open fashion, these body fluid sample collecting regions 110/120/130/140 can also be considered as open body fluid sample collecting regions.

According to an embodiment of the present invention, the body fluid sample collecting regions 110/120/130/140 of different shapes can be configured to collect different body fluid samples (such as blood, tissue fluid, teardrops, urine, saliva and etc.) respectively. Because the viscosities of different body fluids are different, the amount (volume or quantity) of the body fluids filled with the hydrophilic layers 112/122/132/142 will not be the same. Therefore, the quantification required for analysis can be designed to be determined by the hydrophilic layer 112/122/132/142 surface area. The use of different shapes of the body fluid sample collecting regions 110/120/130/140 allows users to easily identify and distinguish the collection regions applicable to different types of body fluid samples. For example, blood is to be collected with a diamond-shaped body fluid sample collecting region 110, and teardrops with a triangular body fluid sample collecting region 120. In addition, for quantitative selection, the same shape body fluid sample collecting regions 110/120/130/140 can also be configured as a plurality of different surface area (not shown), providing users to collect the required quantitative body fluid.

According to another embodiment, the different-shaped body fluid sample collecting regions 110/120/130/140 may be configured to collect different quantitative body fluid samples (eg, 0.1 ml, 0.2 ml, 0.3 ml, etc.). Some studies found that the viscosity values of the same body fluid from different persons are very close, so the quantification required for analysis can be determined solely by the specific surface area of the hydrophilic layer 112/122/132/142 when fully loaded with the same type of body fluid. The use of different shapes of the body fluid sample collecting block 110/120/130/140 allows the user to easily identify and distinguish the body fluid sample collecting regions suitable for collecting different amounts of body fluid. For example, 0.2 ml, 0.1 ml and 0.3 ml of blood are to be collected using diamond-shaped body fluid sample collecting block 110, triangular body fluid sample collecting block 120 and hexagonal body fluid sample collecting block 130 respectively. Users can choose the appropriate body fluid sample collecting region 110/120/130/140 according to the needs of the examination.

According to yet another embodiment, the different body fluid sample collecting regions 110/120/130/140 with different surface areas may be configured to collect different quantitative body fluid samples. For example, users can choose the body fluid sample collecting device with several body fluid collecting regions for different biomarkers with one to more body fluid samples. The different shapes of the body fluid sample collecting block 110/120/130/140 helps to identify the intended usage of the body fluid samples.

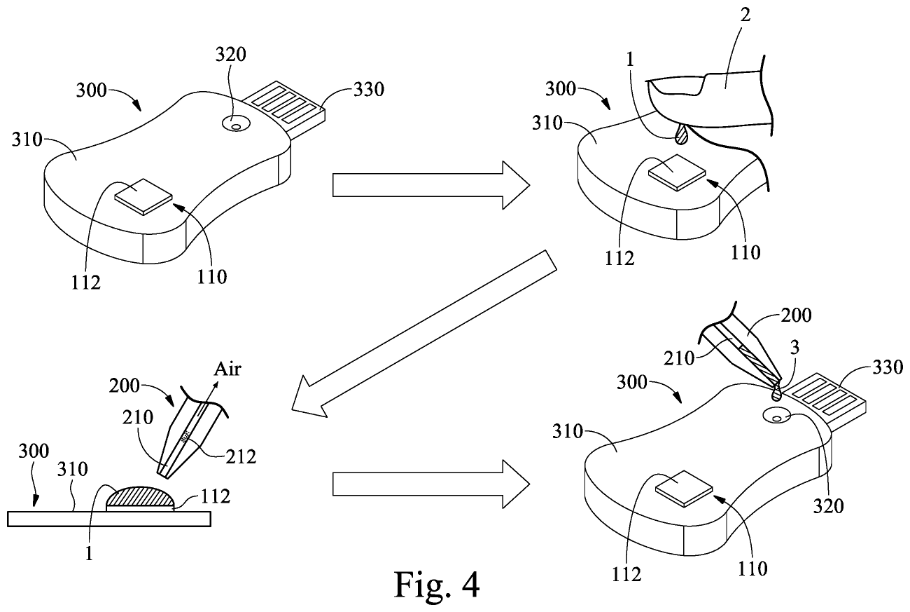

Please refer to FIG. 4, according to another embodiment, which shows a combination from body fluid collecting device to biosensor 300, where the body fluid collecting region 110 was maintained on the sensor surface 310 and the hydrophilic layer 112 was disposed on the body fluid collecting region 110. According to the aforementioned, the body fluid sample 1 fills the hydrophilic layer 112 with specific area on the body fluid collecting region 110, and then the user uses the body fluid sample inspection device 200 with or without reagent 212 to collect and store the body fluid sample 1 through the microchannel 210. During the storage process, the body fluid sample 1 can react with the chemical reagent 212. After a certain period of time, the reaction is completed, one may drop the sample mixture 3 into the sample loading site 320 of the biosensor 300, and plug the biosensing terminal 330 into an appropriate biosensing device (not shown) to identify the test result.

In summary of the aforementioned, the body fluid sample collecting device 100 and the body fluid sample inspection device 200 provided by the present invention constitute a kit, which are respectively used in different stages. In the process of collecting bodily fluids, the bodily fluid sample collecting device 100 can be sterilized in advance, and there is no chemical substance disposed thereon, which can avoid any risk of infection to the person to be examined. Furthermore, it can prevent the body surface of the person to be examined from directly contacting the chemical reagent and avoid the risk of infection by the chemical reagent if a body fluid sample used for inspection is collected from the body fluid sample collecting device 100 to the body fluid sample inspection device 200 containing chemical reagents.

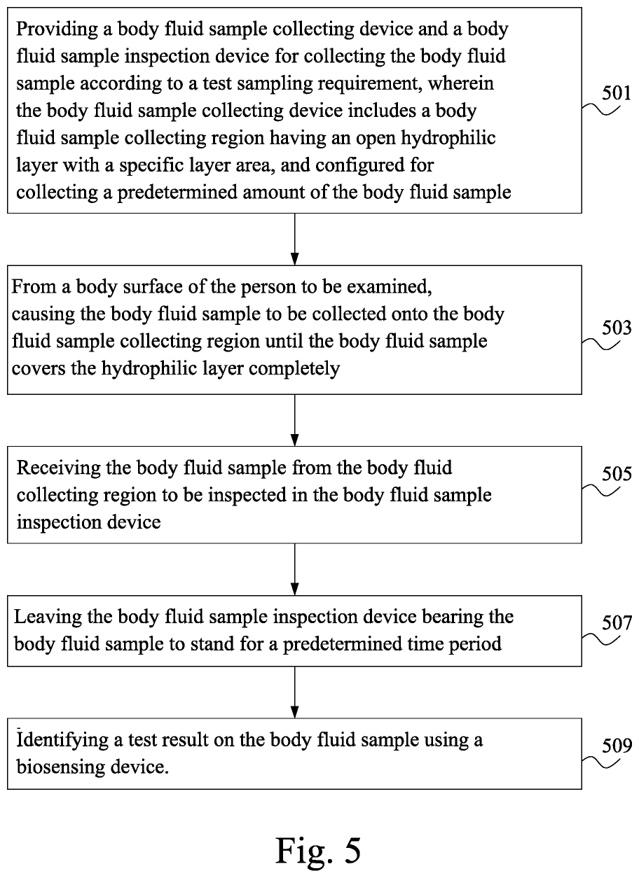

Please refer to FIG. 5, which shows a schematic flow of an embodiment of the method for collecting a body fluid sample of a person to be examined according to the present invention. The method can use the aforementioned device, and follow the steps described below.

First, according to a test sampling requirement, a body fluid sample inspection device and a body fluid sample collecting device having an integrated fluid sample collecting region are selected for the collecting of the body fluid sample, wherein the body fluid sample collecting region has a hydrophilic layer and is configured for collecting a predetermined amount of the body fluid sample (Step 501). The hydrophilic layer is arranged on the body fluid sample collecting region in an open fashion. Body fluid samples can be such as blood, interstitial fluid, teardrops, urine, saliva, and the like. The body fluid sample collecting region can be configured separately on one body fluid sample collecting device, or for the convenience of selection, multiple different body fluid sample collecting regions can also be configured together on one body fluid sample collecting device.

Relying on the person to be examined, the body fluid sample is directly collected from the body surface to the body fluid sample collecting region until the body fluid sample completely covers the hydrophilic layer (Step 503). Referring to FIGS. 2A-2C, it can be realized that, for a specific body fluid sample 1, the hydrophilic layer has a specific surface area, the body fluid sample inspection device 200 has a sample inlet 214 and a microfluidic channel 210 connected to the sample inlet 214, and chemical reagent 212 is arranged in the microfluidic channel 210.

Step 503 may include a sub-step: letting the body surface of the examinee close to any corner location of the body fluid sample collecting region; and causing the body fluid sample to enter the body fluid sample collecting region from the body surface. Once the body fluid sample flows onto the body fluid sample collecting region from a corner thereof, the hydrophilic effect of the hydrophilic layer will continue to spread to all portions of the layer, and finally covers the layer completely. This process does not require the assistance of any other tool, and the person to be examined can complete the process by himself. Therefore, even in a remote area, a person to be examined can easily collect body fluids using the body fluid sample collecting device of the present invention.

After using the body fluid sample collecting device to collect the body fluid sample from the body surface, it may further include the following steps: using the body fluid sample inspection device to collect the body fluid sample from the body fluid sample collecting region and preserve the body fluid sample (step 505), this step can avoid direct contact of the body surface with the chemical reagents in the inspection device and reduce the risk of infection; leaving the body fluid sample inspection device bearing the body fluid sample for a predetermined time (step 507); and using a biosensing device to identify a test result on the body fluid sample (step 509).

Since the body fluid sample collecting device of the present invention is a portable small apparatus, it can be easily transported after the body fluid sample is collected. The body fluid sample inspection device of the present invention is also a small apparatus that is easy to carry, so such a tool kit can be conveniently used in any area. For example, the examinee or user can bring the kit of the invention to a remote area to collect and store different body fluid samples, and then transfer them to a medical institution with a biosensing device for analysis when necessary.

Through the above-mentioned embodiments, the kit and method for collecting body fluid samples of a person to be examined provided by the present invention can provide a convenient and safe technical solution, which can be a major innovation in the art.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A method for collecting a body fluid sample of a person to be examined, comprising steps of:
providing a body fluid sample collecting device and a body fluid sample inspection device for collecting the body fluid sample according to a test sampling requirement, wherein the body fluid sample collecting device includes a body fluid sample collecting region having an open hydrophilic layer with a specific layer area, and configured for collecting a predetermined amount of the body fluid sample;
from a body surface of the person to be examined, causing the body fluid sample to be collected onto the body fluid sample collecting region until the body fluid sample covers the hydrophilic layer completely; and
receiving the body fluid sample from the body fluid collecting region to be inspected in the body fluid sample inspection device or to be preserved.

2. The method according to claim 1, wherein the body fluid sample inspection device includes a sampling inlet and a microfluidic portion connected to the sampling inlet, a reagent is disposed on the microfluidic portion, and the method further comprising steps of:
leaving the body fluid sample inspection device bearing the body fluid sample to stand for a predetermined time period; and
identifying a test result on the body fluid sample using a biosensing device.

3. The method according to claim 1, wherein the reagent includes one or any combination of an antibody, an enzyme, a protein, an infectious substance, a toxic chemical and a heavy metal.

4. The method according to claim 1, wherein the causing step includes a sub-step of:
allowing the body surface of the person to be examined close to any corner position of the body fluid sample collecting region, so that the body fluid sample is caused to enter the body fluid sample collecting region from the body surface.

5. The method according to claim 1, wherein the body fluid sample includes one of a blood, a tissue fluid, a teardrop, a urine and a saliva.

6. A kit for collecting a body fluid sample of a person to be examined, comprising:
a body fluid sample collecting device including a body fluid sample collecting region, wherein the body fluid sample collecting region has an open hydrophilic layer with a specific layer area, and configured for collecting a predetermined amount of the body fluid sample; and
a body fluid sample inspection device including a sampling inlet, a microfluidic portion connected to the sampling inlet and a reagent disposed on the microfluidic portion, and configured to receive therein for inspection the body fluid sample from the body fluid sample collecting region.

7. The kit according to claim 6, wherein the body fluid sample collecting device further includes at least a second body fluid sample collecting region with all regions different from each other, the at least a second body fluid sample collecting regions is each used for collecting a different type of body fluid sample, all the body fluid sample collecting regions on the body fluid sample collecting device have different surface areas, or different shapes, each of which has a particular surface area, and or indicates a particular type of body fluid sample to be collected therein.

8. The kit according to claim 6, wherein the body fluid sample collecting region has a first shape identifying intention of detecting a first biomarker, and the body fluid sample collecting device further includes a second body fluid sample collecting region having a different for a second shape identifying intention of detecting a second biomarker.

9. The kit according to claim 6, wherein the reagent includes one or any combination of an antibody, an enzyme, a protein, an infectious substance, a toxic chemical and a heavy metal.

10. The kit according to claim 6, wherein the body fluid sample includes one of a blood, a tissue fluid, a teardrop, a urine and a saliva.

11. The kit according to claim 6, wherein the body fluid collecting device can be combined into a biosensor by using the body fluid sample inspection device to transfer the sample to be examined into the biosensor, and using the biosensing device to analyze.

12. A method for collecting a body fluid sample of a person to be examined, comprising steps of:
selecting a body fluid sample collecting device and a body fluid sample inspection device for collecting the body fluid sample according to a test sampling requirement, wherein the body fluid sample collecting device includes a body fluid sample collecting region having an open hydrophilic layer with a specific layer area, and configured for collecting a predetermined amount of the body fluid sample;
from a body surface of the person to be examined, causing the body fluid sample to be collected onto the body fluid sample collecting region until the body fluid sample covers the hydrophilic layer completely;
receiving the body fluid sample from the body fluid collecting region to be inspected in the body fluid sample inspection device;
wherein the body fluid sample inspection device includes a sampling inlet and a microfluidic portion connected to the sampling inlet, a reagent is disposed on the microfluidic portion; and leaving the body fluid sample inspection device bearing the body fluid sample to stand for a predetermined time period; and
identifying a test result on the body fluid sample using a biosensing device.

13. A method according to claim 11, wherein the body fluid sample collecting device is a biosensor having a sample loading site, and the method further comprising a step of dropping the body fluid sample into the sample loading site before identifying a test result on the body fluid sample using a biosensing device.
